(19) United States
(12) Patent Application Publication
(10) Pub. No.: US 2020/0003364 A1
(43) Pub. Date: Jan. 2, 2020

(54) FLUID CONTAINER REFILLING SYSTEM

(71) Applicant: Boston Scientific Scimed Inc, Maple Grove, MN (US)

(72) Inventors: Chadi Harmouche, Quebec (CA); Eric Ryba, San Diego, CA (US)

(21) Appl. No.: 16/567,312

(22) Filed: Sep. 11, 2019

Related U.S. Application Data
(63) Continuation of application No. PCT/US2018/021821, filed on Mar. 9, 2018.
(60) Provisional application No. 62/470,636, filed on Mar. 13, 2017.

(51) Int. Cl. F17C 5/04; A61B 18/02

(52) U.S. Cl. CPC F17C 5/04; A61B 18/02; F17C 2223/0123; F17C 2225/013; F17C 2270/02; F17C 2227/0341; F17C 2227/04; F17C 2205/0341; F17C 2265/012; F17C 2227/0157

(57) ABSTRACT

A method for filling a fluid container with a cryogenic fluid, the method including the step of reducing with a container temperature reducer a container temperature of the fluid container prior to adding the cryogenic fluid to the fluid container. The method can include additional steps, including: compressing with a compressor the cryogenic fluid based at least partially on the container temperature, controlling with a controller the extent that the compressor compresses the cryogenic fluid, cooling with a fin pack the cryogenic fluid, filtering with a filter the cryogenic fluid, analyzing with a fluid analyzer a fluid within the cryogenic fluid, monitoring with a water monitor a water content of the cryogenic fluid and/or monitoring with a fluid sensor a fluid property of the cryogenic fluid.

### CROSS-REFERENCE TO RELATED APPLICATION

This application is a continuation of International Application No. PCT/US2018/021821, with an international filing date of Mar. 9, 2018, which claims the benefit of U.S. Provisional Application No. 62/470,636, filed Mar. 13, 2017, and entitled “REDUCED PRESSURE MEDICAL COOLING FLUID REFILLING ASSEMBLY AND METHOD”, which is herein incorporated by reference in its entirety.

### TECHNICAL FIELD

The present invention relates to medical devices and methods for cryoablation. More specifically, the invention relates to devices and methods for delivering cryogenic fluid to a cryoablation system fluid source.

### BACKGROUND

Cardiac arrhythmias involve an abnormality in the electrical conduction of the heart and are a leading cause of stroke, heart disease, and sudden cardiac death. Treatment options for patients with arrhythmias include medications and/or the use of medical devices, which can include implantable devices and/or catheter ablation of cardiac tissue, to name a few. In particular, catheter ablation involves delivering ablative energy to tissue inside the heart to block aberrant electrical activity from depolarizing heart muscle cells out of synchrony with the heart's normal conduction pattern. The procedure is performed by positioning the tip of an energy delivery catheter adjacent to diseased or targeted tissue in the heart. The energy delivery component of the system is typically at or near the most distal (i.e. farthest from the operator or user) portion of the catheter, and often at the tip of the catheter.

Various forms of energy can be used to ablate diseased heart tissue. One form of energy that is used to ablate diseased heart tissue includes cryogenics (also referred to herein as “cryoablation”). During a cryoablation procedure, the tip of the catheter is positioned adjacent to targeted cardiac tissue, at which time energy is delivered in the form of a refrigerant or cryogenic fluid to create tissue necrosis, rendering the ablated tissue incapable of conducting electrical signals.

Cryosurgical, and in particular, catheter-based cryoablation systems consume various cryogenic fluids (e.g., liquid nitrous oxide or liquid nitrogen) that are typically provided in high-pressure fluid containers in either liquid or gas form (collectively referred to herein as “cryogenic fluid”). During cryoablation procedures, one or more alternative fluid containers may be used to contain the cryogenic fluid. When the level of cryogenic fluid within the fluid container is determined to be below a certain predetermined level, it is understood that such fluid container needs to be refilled with cryogenic fluid from a fluid supply source, i.e., at a hospital or other patient treatment location. When it is determined that it is an appropriate time to refill the fluid container, it is desired that the filling and/or refilling of the fluid container be accomplished in a safe, efficient and effective manner.

Current proposed methods of providing the necessary supply of the cryogenic fluid from the fluid supply source have various shortcomings. One proposed method depends on the need to compress and filter the cryogenic fluid from the fluid supply source to increase the pressure such that the cryogenic fluid can be liquefied. However, this proposal does not provide any clear method by which this can be achieved. The proposed requirements fall outside the capabilities of “off-the-shelf” pumps or filters. For example, compressing a gas from 500 to 1000 psi would require a multi-stage pump, which itself can be a major source of contamination to the final compressed cryogenic fluid, which needs to be very clean for use in the catheter-based cryoablation systems. Another proposed method relies on subcooling the cryogenic fluid such that the cryogenic fluid can be liquefied. However, even with subcooling, in order to liquefy the cryogenic fluid, the pumps or filters will undergo significant wear-and-tear.

### SUMMARY

The present invention is directed toward a method for filling a fluid container with a cryogenic fluid, the method including the steps of reducing with a container temperature reducer a container temperature of the fluid container from a first temperature to a second temperature prior to adding the cryogenic fluid to the fluid container. The step of reducing can include reducing the second temperature to at least approximately 10° C. lower than the first temperature.

In some embodiments, the method can further include the step of compressing with a compressor the cryogenic fluid based at least partially on the container temperature of the fluid container.

In certain embodiments, the method can also include the step of controlling with a controller the extent that the compressor compresses the cryogenic fluid based at least partially on the container temperature of the fluid container. The step of controlling can further include controlling the container temperature reducer to adjust the container temperature of the fluid container.

In various embodiments, the method can also include the step of cooling with a fin pack the cryogenic fluid that exits the compressor.

In certain embodiments, the method can also include the step of filtering with at least one filter the cryogenic fluid after the cryogenic fluid is compressed by the compressor. The filter can include at least one of a replaceable cartridge and a regenerating cartridge.

In some embodiments, the method can further include the step of selectively heating with a filter heater the filter.

In various embodiments, the method can also include the step of pulling with a vacuum moisture from the filter.

In certain embodiments, the method can also include the step of analyzing with a fluid analyzer a fluid within the cryogenic fluid after the cryogenic fluid is compressed by the compressor. The fluid analyzer can be positioned downstream from at least one or more filters.

In various embodiments, the method can also include the step of monitoring with a water monitor a water content of the cryogenic fluid after the cryogenic fluid is compressed by the compressor. The water monitor can be connected to the controller, the controller being configured to alert an operator when the water content exceeds a predetermined threshold level.

In some embodiments, the method can further include the step of monitoring with a fluid sensor a fluid property of the cryogenic fluid. The fluid property of the cryogenic fluid can include one of a fluid pressure and a fluid temperature. In such embodiments, the step of monitoring can include monitoring the fluid property of the cryogenic fluid within the fluid container. Alternatively, the step of monitoring can include monitoring the fluid property of the cryogenic fluid prior to adding the cryogenic fluid to the fluid container.

Further, in certain applications, the present invention is directed toward a fluid container refilling system (also sometimes referred to as a “container refilling system”) for filling a fluid container with a cryogenic fluid from a fluid supply source, the container refilling system including a container temperature reducer that can be configured to reduce a container temperature of the fluid container from a first temperature to a second temperature prior to adding the cryogenic fluid to the fluid container.

In certain embodiments, the container refilling system can also include a compressor that is configured to compress the cryogenic fluid based at least partially on the container temperature of the fluid container.

In some embodiments, the container refilling system can also include a controller that controls the extent that the compressor compresses the cryogenic fluid based at least partially on the container temperature of the fluid container.

In various embodiments, the container refilling system can also include a fin pack that is configured to cool the cryogenic fluid that exits the compressor.

In certain embodiments, the container refilling system can also include a filter that is configured to filter the cryogenic fluid after the cryogenic fluid is compressed by the compressor.

In some embodiments, the container refilling system can also include a filter heater that is configured to selectively heat the filter. In various embodiments, the container refilling system can also include a vacuum that is configured to pull moisture from the filter.

In various embodiments, the container refilling system can also include a vacuum that is configured to pull moisture from the filter.

In certain embodiments, the container refilling system can also include a gas analyzer that is configured to analyze a gas within the cryogenic fluid after the cryogenic fluid is compressed by the compressor.

In some embodiments, the container refilling system can also include a water monitor that is configured to monitor a water content of the cryogenic fluid after the cryogenic fluid is compressed by the compressor.

In various embodiments, the container refilling system can also include a fluid sensor that is configured to monitor a fluid property of the cryogenic fluid. In one embodiment, the fluid sensor can monitor the fluid property of the cryogenic fluid prior to adding the cryogenic fluid to the fluid container. In another embodiment, the fluid sensor can monitor the fluid property of the cryogenic fluid within the fluid container.

### BRIEF DESCRIPTION OF THE DRAWINGS

The novel features of this disclosure, both as to structure and operation, will be best understood from the accompanying drawings, taken in conjunction with the accompanying description, in which similar reference characters refer to similar parts, and in which:

FIG. 1 is a schematic view of a patient, a fluid supply source and one embodiment of the cryogenic balloon catheter system including a fluid container refilling system; and

FIG. 2 is a simplified schematic side view of the fluid supply source with the cryogenic fluid, the fluid container with the cryogenic fluid, and one embodiment of the fluid container refilling system.

### DETAILED DESCRIPTION

Embodiments of the present invention are described herein in the context of a fluid container refilling system (also sometimes referred to herein as a “container refilling system”), and corresponding methods, which are usable with a suitable ablation system and/or catheter system. In particular, as provided in detail herein, the container refilling system can include various features that enable the operator or user to fill or refill the fluid container with cryogenic fluid in a more safe, convenient and effective manner.

Those of ordinary skill in the art will realize that the following detailed description of the present invention is illustrative only and is not intended to be in any way limiting. Other embodiments of the container refilling system will readily suggest themselves to such skilled persons having the benefit of this disclosure. Reference will now be made in detail to implementations of the present invention as illustrated in the accompanying drawings.

In the interest of clarity, not all of the routine features of the implementations described herein are shown and described. It will, of course, be appreciated that in the development of any such actual implementation, numerous implementation-specific decisions must be made in order to achieve the developer's specific goals, such as compliance with application-related and business-related constraints, and that these specific goals will vary from one implementation to another and from one developer to another. Moreover, it will be appreciated that such a development effort might be complex and time-consuming, but would nevertheless be a routine undertaking of engineering for those of ordinary skill in the art having the benefit of this disclosure.

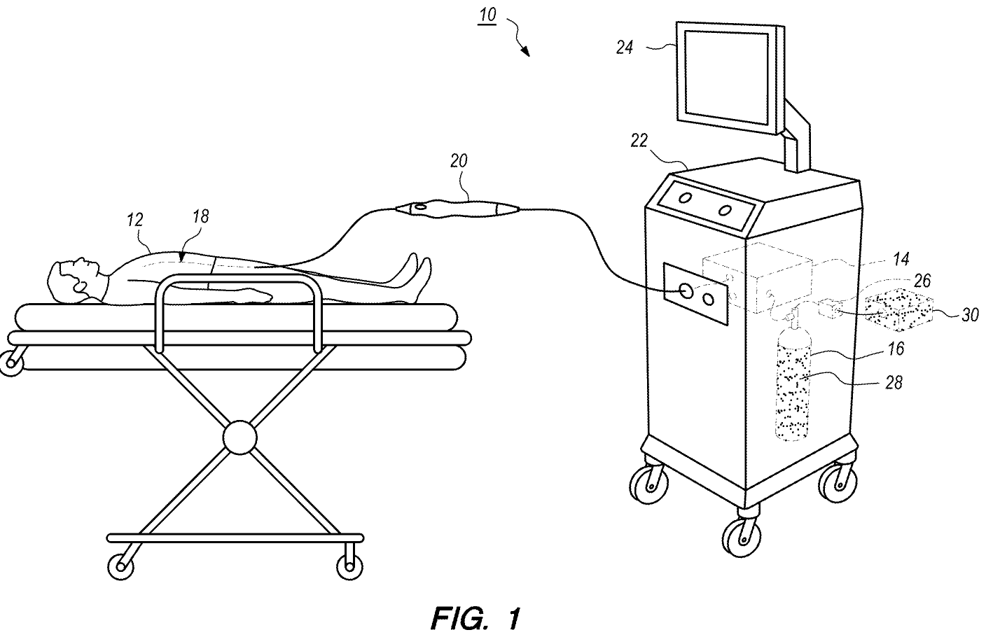

FIG. 1 is a schematic view of one embodiment of a cryogenic balloon catheter system 10 (also sometimes referred to as a “catheter system”) for use with a patient 12, which can be a human being or an animal. Although the catheter system 10 is specifically described herein with respect to the cryogenic balloon catheter system, it is understood and appreciated that other types of catheter systems and/or ablation systems can equally benefit by the teachings provided herein. For example, in certain non-exclusive alternative embodiments, the present invention can be equally applicable for use with any suitable types of ablation systems and/or any suitable types of catheter systems. Thus, the specific reference herein to use as part of the cryogenic balloon catheter system is not intended to be limiting in any manner.

The design of the catheter system 10 can be varied. In certain embodiments, such as the embodiment illustrated in FIG. 1, the catheter system 10 can include one or more of a control system 14, a fluid source 16 (e.g., one or more fluid containers), a balloon catheter 18, a handle assembly 20, a control console 22, a graphical display 24 (also sometimes referred to as a graphical user interface or “GUI”) and a container refilling system 26. It is understood that although FIG. 1 illustrates the structures of the catheter system 10 in a particular position, sequence and/or order, these structures can be located in any suitably different position, sequence and/or order than that illustrated in FIG. 1. It is also understood that the catheter system 10 can include fewer or additional components than those specifically illustrated and described herein.

In various embodiments, the control system 14 is configured to monitor and control the various processes of the ablation procedure. More specifically, the control system 14 can monitor and control release and/or retrieval of a cryogenic fluid 28 to and/or from the balloon catheter 18. The control system 14 can also control various structures that are responsible for maintaining and/or adjusting a flow rate and/or pressure of the cryogenic fluid 28 that is released to the balloon catheter 18 during the cryoablation procedure. In such embodiments, the catheter system 10 delivers ablative energy in the form of cryogenic fluid 28 to cardiac tissue of the patient 12 to create tissue necrosis, rendering the ablated tissue incapable of conducting electrical signals. Additionally, in various embodiments, the control system 14 can control activation and/or deactivation of one or more other processes of the balloon catheter 18. Further, or in the alternative, the control system 14 can receive data and/or other information (also sometimes referred to as “sensor output”) from various structures within the catheter system 10. In various embodiments, the control system 14 and the GUI 24 and/or the container refilling system 26 can be electrically connected and/or coupled. In some embodiments, the control system 14 can receive, monitor, assimilate and/or integrate any sensor output and/or any other data or information received from any structure within the catheter system 10 in order to control the operation of the balloon catheter 18. Still further, or in the alternative, the control system 14 can control positioning of portions of the balloon catheter 18 within the body of the patient 12, and/or can control any other suitable functions of the balloon catheter 18.

The fluid source 16 (also sometimes referred to as “fluid container 16”) can include one or more fluid container(s) 16. It is understood that while one fluid container 16 is illustrated in FIG. 1, any suitable number of fluid containers 16 may be used. The fluid container(s) 16 can be of any suitable size, shape and/or design. The fluid container(s) 16 contains the cryogenic fluid 28, which is delivered to the balloon catheter 18 with or without input from the control system 14 during a cryoablation procedure. Additionally, the type of cryogenic fluid 28 that is used during the cryoablation procedure can vary. In one non-exclusive embodiment, the cryogenic fluid 28 can include liquid nitrous oxide. In another non-exclusive embodiment, the cryogenic fluid 28 can include liquid nitrogen. However, any other suitable cryogenic fluid 28 can be used.

The design of the balloon catheter 18 can be varied to suit the specific design requirements of the catheter system 10. As shown, the balloon catheter 18 is inserted into the body of the patient 12 during the cryoablation procedure. In one embodiment, the balloon catheter 18 can be positioned within the body of the patient 12 using the control system 14. Stated in another manner, the control system 14 can control positioning of the balloon catheter 18 within the body of the patient 12. Alternatively, the balloon catheter 18 can be manually positioned within the body of the patient 12 by a qualified health professional (also referred to herein as an “operator” or “user”). As used herein, health care professional, operator and/or user can include a physician, a physician's assistant, a nurse and/or any other suitable person and/or individual. In certain embodiments, the balloon catheter 18 is positioned within the body of the patient 12 utilizing at least a portion of the sensor output that is received from the balloon catheter 18. For example, in various embodiments, the sensor output is received by the control system 14, which can then provide the operator with information regarding the positioning of the balloon catheter 18. Based at least partially on the sensor output feedback received by the control system 14, the operator can adjust the positioning of the balloon catheter 18 within the body of the patient 12 to ensure that the balloon catheter 18 is properly positioned relative to targeted cardiac tissue. While specific reference is made herein to the balloon catheter 18, as noted above, it is understood that any suitable type of medical device and/or catheter may be used.

The handle assembly 20 is handled and used by the operator or user to operate, position and control the balloon catheter 18. The design and specific features of the handle assembly 20 can vary to suit the design requirements of the catheter system 10. In the embodiment illustrated in FIG. 1, the handle assembly 20 is separate from, but in electrical and/or fluid communication with the control system 14, the fluid container 16, the GUI 24 and/or the container refilling system 26. In some embodiments, the handle assembly 20 can integrate and/or include at least a portion of the control system 14 within an interior of the handle assembly 20. It is understood that the handle assembly 20 can include fewer or additional components than those specifically illustrated and described herein.

In the embodiment illustrated in FIG. 1, the control console 22 includes at least a portion of the control system 14, the fluid container 16, the GUI 24 and/or the container refilling system 26. However, in alternative embodiments, the control console 22 can contain additional structures not shown or described herein. Still alternatively, the control console 22 may not include various structures that are illustrated within the control console 22 in FIG. 1. For example, in certain nonexclusive alternative embodiments, the control console 22 does not include the GUI 24.

In various embodiments, the GUI 24 is electrically connected to the control system 14 and/or the container refilling system 26. Additionally, the GUI 24 provides the operator or user of the catheter system 10 with information that can be used before, during and after the cryoablation procedure. For example, the GUI 24 can provide the operator or user with information based on the sensor output, and any other relevant information that can be used before, during and after the cryoablation procedure. The specifics of the GUI 24 can vary depending upon the design requirements of the catheter system 10, or the specific needs, specifications and/or desires of the operator or user.

In one embodiment, the GUI 24 can provide static visual data and/or information to the operator or user. In addition, or in the alternative, the GUI 24 can provide dynamic visual data and/or information to the operator or user, such as video data or any other data that changes over time, e.g., during an ablation procedure. Further, in various embodiments, the GUI 24 can include one or more colors, different sizes, varying brightness, etc., that may act as alerts to the operator or user. Additionally, or in the alternative, the GUI 24 can provide audio data or information to the operator or user.

The container refilling system 26 fills and/or refills fluid container(s) 16 with cryogenic fluid 28 that is then used with the catheter system 10. The container refilling system 26 receives the cryogenic fluid 28 from a fluid supply source 30, typically in a gaseous state. The type of cryogenic fluid 28 that is used can vary. As used herein, “fluid supply source 30” can include a hospital or other health care facility gas supply, as non-exclusive examples (sometimes referred to herein as “facility gas supply”). Alternatively, the fluid supply source 30 can include any other suitable gas supply.

As described herein, the container refilling system 26 processes a gaseous cryogenic fluid 28 from the fluid supply source 30 into a liquid cryogenic fluid 28, which is used to fill the fluid container(s) 16. It is understood that the gaseous cryogenic fluid 28 from the fluid supply source 30 undergoes certain processing along portions of the container refilling system 26 until the cryogenic fluid 28 enters the fluid container 16.

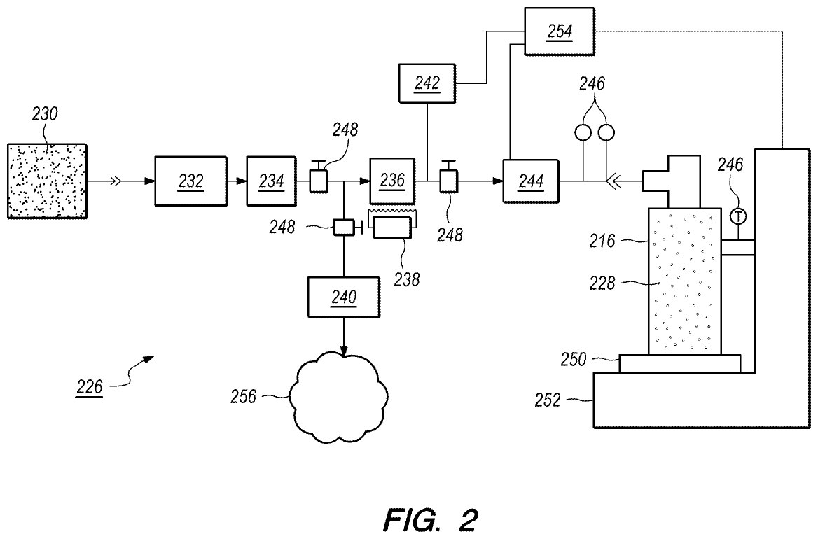

In the embodiment illustrated in FIG. 1, at least a portion of the container refilling system 26 is positioned at a location within the control console 22. The container refilling system 26 can be positioned at any suitable location within the control console 22. Further, portions of the container refilling system 26 can be positioned partially within and/or outside the control console 22. Alternatively, the container refilling system 26 can be positioned at any suitable location outside of the control console 22. Additionally, and/or alternatively, the container refilling system 26 can be positioned at any other suitable location within the catheter system 10. In various embodiments, at least a portion of the container refiling system 26 can be electrically connected and/or coupled to the control system 14 and/or the GUI 24. The specific components and operations of the container refilling system 26 will be described in greater detail herein below in relation to the embodiment illustrated in FIG. 2.

FIG. 2 is a simplified schematic view of the fluid supply source 230 with the cryogenic fluid 228, the fluid container 216 with the cryogenic fluid 228, and another embodiment of the container refilling system 226. The design of the container refilling system 226 can be varied. In one embodiment, the container refilling system 226 can include one or more of a compressor 232, a fin pack 234, one or more filters 236, a filter heater 238, a vacuum 240, a fluid analyzer 242, a water monitor 244, one or more fluid sensors 246, one or more isolation valves 248, a scale 250, a container temperature reducer 252 and a controller 254. It is understood that although the embodiment illustrated in FIG. 2 includes various structures, not all of these structures are required in every embodiment of the container refilling system 226. For example, in some embodiments, one or more structures illustrated in FIG. 2 can be omitted without substantially deviating from the intent of the container refilling system 226. Moreover, in other embodiments, one or more additional structures that are not illustrated in FIG. 2 can be included without substantially deviating from the intent of the container refilling system 226.

The compressor 232 receives and compresses the gaseous cryogenic fluid 228 from the fluid supply source 230. The act of compression will heat the cryogenic fluid 228 considerably. The design of the compressor 232 can vary to suit the design requirements of the container refilling system 226 and/or the catheter system 10 (illustrated in FIG. 1). In one embodiment, the compressor 232 may be of the oil-free variety in order to minimize further contamination of the cryogenic fluid 228 being compressed. In another embodiment, the compressor 232 may be a multistage compressor in order to achieve the higher compression ratios, as needed. Alternatively, any variety of compressor 232 may be used.

The fin pack 234 blows ambient air over finned tubing that carries the compressed (now heated) cryogenic fluid 228 in order to cool the compressed cryogenic fluid 228. The fin pack 234 can be of any suitable design that enables the fin pack 234 to effectively cool the cryogenic fluid 228. Additionally, the fin pack 234 can function to cool the cryogenic fluid 228 via any suitable manner and/or method.

The one or more filters 236 receive the cryogenic fluid 228 after the fin pack 234 has at least partially cooled the compressed cryogenic fluid 228. The design and/or configuration of the filters 236 can be varied. In one embodiment, at least one of the filters 236 can be a replaceable cartridge-type filter that is replaced/replenished when full. Alternatively, at least one of the filters 236 can be of a regenerating cartridge-type that can be regenerated in place through the steps of evacuating and heating to draw off the moisture that has been trapped within the filter 236 during a drying process. In some embodiments, at least one of the filters 236 can be positioned such that the cooling of the fluid container 216 cools the filter 236. In other embodiments, the capacity of at least one of the filters 236 can be increased by operating it at a lower temperature. In certain embodiments, at least one of the filters 236 can be of the molecular sieve variety that preferentially filters molecules of a specific size from the cryogenic fluid 228 moving through the filter 236. More specifically, at least one of the filters 236 can be of a 3A or a 4A type, as nonexclusive examples. Alternatively, at least one of the filters 236 can filter particles of a greater and/or a lesser size. Additionally, and/or in the alternative, one or more of the filters 236 can absorb certain particles.

In certain embodiments, the filter heater 238 can selectively heat the filter 236 to draw off moisture that has been trapped within the filter 236 during the drying process. The filter heater 238 can be of any suitable design that enables the filter heater 238 to effectively heat the filter 236 to draw off moisture that has been trapped within the filter 236. Additionally, the filter heater 238 can function to heat the filter 236 via any suitable manner and/or method.

In various embodiments, the vacuum 240 can be used in conjunction with the heater 238 to remove collected moisture from the filter 236. The filter 236 can have limited capacity so the filter 236 can either be replaced regularly or purged (regenerated) occasionally. Using the filter heater 238 and the vacuum 240 are effective methods to remove materials collected by the one or more filters 236. In some embodiments, the vacuum 240 can expel any collected moisture in the form of exhaust 256 to a safe area. The design of the vacuum 240 can vary. In one embodiment, the vacuum 240 can include a low pressure sensor (not shown) that monitors a pressure of the vacuum 240 as the filter 236 is regenerated. Alternatively, the vacuum 240 can be of any suitable design that allows the vacuum 240 to remove collected moisture from the filter 236. Additionally, the vacuum 240 can function to remove collected moisture from the filter 236 via any suitable manner and/or method.

The fluid analyzer 242 analyzes the at least partially processed cryogenic fluid 228. The fluid analyzer 242 can be of any suitable design that enables the fluid analyzer 242 to effectively analyze the at least partially processed cryogenic fluid 228. Additionally, the fluid analyzer 242 can function to analyze the at least partially processed cryogenic fluid 228 via any suitable manner and/or method. In the embodiment illustrated in FIG. 2, the fluid analyzer 242 is positioned downstream from the filter 236 in order to increase the likelihood that the proper purity level is being achieved. However, it is recognized that the fluid analyzer 242 can be positioned in a different location that is either upstream from the filter 236, or further downstream than what is illustrated in FIG. 2. Additionally, in another embodiment, greater than one fluid analyzer 242 can be used in different locations from one another within the container refilling system 226. In one embodiment, the fluid analyzer 242 can be connected to the controller 254 in a fashion that alerts an operator or user when it becomes necessary to perform service on the container refilling system 226 due to the eventual degradation of one or more of the filters 236, for example, or for other suitable reasons.

The water monitor 244 monitors water content of the at least partially processed cryogenic fluid 228 following filtration by the filter(s) 236. The water monitor 244 can be of any suitable design that enables the water monitor 244 to effectively monitor the at least partially processed cryogenic fluid 228. Additionally, the water monitor 244 can function to monitor the at least partially processed cryogenic fluid 228 via any suitable manner and/or method. For example, in one embodiment, the water monitor 244 can be connected to the controller 254 in a manner that alerts the operator or user when the water content of the at least partially processed cryogenic fluid 228 exceeds a predetermined threshold level. The predetermined threshold level can include any suitable level as determined by the operator or user.

The fluid sensors 246 can monitor various properties of the cryogenic fluid 228 that is being moved from the fluid supply source 230 along the container refilling system 226. Additionally, the fluid sensors 246 can monitor various properties of the cryogenic fluid 228 within the fluid container 216. The fluid sensors 246 can include one or more of a pressure sensor and/or a temperature sensor. It is understood that although the fluid sensors 246 illustrated in FIG. 2 are shown in certain locations within the container refilling system 226, the fluid sensors 246 can be positioned at any suitable location within the container refilling system 226, including other than those illustrated in FIG. 2. In certain embodiments, the pressure sensor 246 can be positioned adjacent to the fluid container 216 in order to monitor the fluid pressure immediately prior to the processed cryogenic fluid 228 entering the fluid container 216. In some embodiments, the pressure sensor 246 can monitor the fluid pressure of the cryogenic fluid 228 within the fluid container 216. In other embodiments, the temperature sensor 246 can be positioned adjacent to the fluid container 216 in order to monitor the temperature of the processed cryogenic fluid 228 prior to entering the fluid container 216 and/or the cryogenic fluid 228 within the fluid container 216. In various embodiments, the temperature sensor 246 can monitor the temperature of the cryogenic fluid 228 within the fluid container 216 and/or the cryogenic fluid 228 at various stages during the filling/refilling process. Additionally, or in the alternative, the temperature sensor 246 can monitor the temperature of the one or more filters 236 for the purpose of regenerating the one or more filters 236.

One or more isolation valves 248 can be used as needed to isolate portions of the container refilling system 226. In one non-exclusive example, certain isolation valves 248 can be closed, while other isolation valves 248 are opened in order to operate the vacuum 240 as described previously herein, i.e., to remove collected moisture from the one or more filters 236.

The scale 250 monitors the weight of the fluid container 216 and the cryogenic fluid 228 to determine the extent that the fluid container 216 is full during the filling/refilling process. The scale 250 can be of any suitable design that enables the scale 250 to effectively determine whether the fluid container 216 is full or substantially full. Additionally, the scale 250 can function to monitor the weight of the fluid container 216 via any suitable manner and/or method.

The container temperature reducer 252 reduces a container temperature of the fluid container 216 from a first temperature to a second temperature prior to adding the cryogenic fluid 228 to the fluid container 216. As used herein, the container temperature of the fluid container can include the first temperature and the second temperature. The first temperature can include any suitable temperature, which may include room or ambient temperature or some other temperature. Further, the second temperature, can include a temperature that is at least approximately lower than the first temperature. For example, in some embodiments, the second temperature can be at least approximately 10° C., 25° C., 50° C. or 100° C. lower than the first temperature. Alternatively, the second temperature can be at least approximately lower than the first temperature by any other suitable temperature. The container temperature reducer 252 can function to reduce the container temperature of the fluid container 216 from the first temperature to the second temperature via any suitable manner and/or method.

In various embodiments, the container temperature reducer reduces the container temperature of the fluid container 216 from the first temperature to the second temperature in preparation for the fluid container 216 to receive the cryogenic fluid 228. The container temperature reducer 252, by cooling the fluid container 216 from the first temperature to the second temperature, dictates the output pressure the compressor 232 will need to achieve in order to liquefy the gaseous cryogenic fluid 228. For instance, to compress and liquefy gaseous nitrous oxide at the container temperature of the fluid container 216 having the first temperature, including room or ambient temperatures (such as the range of approximately 15° C. to 25° C.), requires that the gaseous nitrous oxide be compressed to a pressure of approximately 750 psi or more. If the container temperature of the fluid container 216 is cooled to the second temperature of 0° C., the gaseous nitrous oxide only needs to be compressed to approximately 435 psi in order for the gaseous nitrous oxide to liquefy. Once the fluid container 216 is filled with the cryogenic fluid 228, the internal pressure within the fluid container 216 will increase to approximately 750 psi as the container temperature of the fluid container 216 increases or warms to the room or ambient temperature. Cooling the container temperature of the fluid container 216 to lower temperatures, i.e., from the first temperature to the second temperature, during filling and/or refilling will allow even lower compression pressures to be used. And once filled with the cryogenic fluid 228, the internal pressure within the fluid container 216 will again return to approximately 750 psi when the container temperature of the fluid container 216 warms to room or ambient temperature. For instance, at −20° C., the compressor 232 would only need to raise the pressure to approximately 230 psi.

The design of the container temperature reducer 252 can vary based on the design requirements of the container refilling system 226 and/or the catheter system 10. In certain embodiments, the container temperature reducer 252 can be a vapor-compression refrigeration system. In one embodiment, the container temperature reducer 252 can be built into a cabinet or other housing that could also contain the fluid container 216, such as the control console 22 (illustrated in FIG. 1). In this embodiment, cold air (or other gas) can be blown over the fluid container 216 to cool the fluid container 216 and/or remove the heat left from the condensation of nitrous oxide as the nitrous oxide condenses from a gas to a liquid.

Once the fluid container 216 is filled with the cryogenic fluid 228, the fluid container 216 would be removed from the container temperature reducer 252 and placed in an ambient environment such that the container temperature of the fluid container 216 can return to ambient temperature before use. As the container temperature of the fluid container 216 increases, the internal pressure within the fluid container 216 can return to the working pressure of approximately 750 psi which is generally what is expected for the working pressure of the catheter system 10.

The controller 254 can be electrically connected to one or more of the structures of the container refilling system 226. As such, the controller 254 can also receive and/or process data from one or more of the structures described herein. In some embodiments, the controller 254 can control the extent that the compressor 232 compresses the cryogenic fluid 228 based at least partially upon the data received and/or processed by the controller 254. As one non-exclusive example, the controller 254 can receive and/or process data regarding the container temperature of the fluid container 216, and can change and/or adjust the extent that the compressor 232 compresses the cryogenic fluid 228 based upon this data. Additionally, and/or in the alternative, the controller 254 can control the container temperature reducer 252 in order to adjust the container temperature of the fluid container 216 based at least partially upon any relevant data received and/or processed by the controller 254.

One advantage of the container refilling system 226 disclosed herein is the ability to more effectively reduce the pressure at which liquefaction occurs by physically cooling or lowering the container temperature of the fluid container 216. The container refilling system 226 disclosed herein will relatively reduce the work needed to liquefy the cryogenic fluid 228 (because of the lower pressure requirement), which can reduce the heat of compression. Reducing the heat of compression in turn can reduce the wear-and-tear on the compressor 232, and thus increase the likelihood of extending the life of the compressor 232.

While a number of exemplary aspects and embodiments of the container refilling system have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

We claim:

1. A method for filling a fluid container of a cryoablation system with a cryogenic fluid, the method comprising the steps of:
reducing a container temperature of the fluid container from a first temperature to a second temperature prior to adding the cryogenic fluid to the fluid container;
compressing the cryogenic fluid, wherein an extent of compression of the cryogenic fluid is based at least partially on the container temperature of the fluid container;
passing the compressed cryogenic fluid through a cooler so as to cool the compressed cryogenic fluid;
passing the cooled and compressed cryogenic fluid through a filter;
delivering the filtered, cooled and compressed cryogenic fluid to the fluid container; and
after delivering the filtered, cooled and compressed cryogenic fluid to the fluid container, selectively heating the filter to remove moisture from therefrom.

2. The method of claim 1, wherein the second temperature is at least approximately 10° C. lower than the first temperature.

3. The method of claim 1, wherein the cooler is a fin pack cooler.

4. The method of claim 1, wherein the filter includes at least one of a replaceable cartridge and a regenerating cartridge.

5. The method of claim 1, further comprising removing collected moisture from the filter under a vacuum.

6. The method of claim 1, further comprising analyzing, with a fluid analyzer, the compressed and cooled cryogenic fluid.

8. The method of claim 6, wherein the fluid analyzer is positioned downstream from the filter.

9. The method of claim 1, further comprising monitoring a water content of the compressed and cooled cryogenic fluid using a water monitor.

10. The method of claim 9, wherein the water monitor is operatively coupled to a controller, the method further comprising the controller generating an alert to an operator when the water content exceeds a predetermined threshold level.

11. The method of claim 1, further comprising the monitoring with a fluid sensor at least one of a fluid pressure or a fluid pressure of the cryogenic fluid.

12. The method of claim 11, wherein monitoring the fluid property of the cryogenic fluid is performed prior to delivering the cryogenic fluid to the fluid container.

13. A container refilling system for filling a fluid container with a cryogenic fluid, the cryogenic fluid being stored within a fluid supply source, the container refilling system comprising:
a container temperature reducer that is configured to reduce a container temperature of the fluid container from a first temperature to a second temperature prior to adding the cryogenic fluid to the fluid container;
a compressor configured to compress the cryogenic fluid based at least partially on the container temperature of the fluid container;
a cooler positioned downstream of the compressor and configured to cool the cryogenic fluid after being compressed;
a filter positioned downstream of the cooler and configured to filter the compressed and cooled cryogenic fluid; and
a filter heater configured to selectively heat the filter to facilitate removal of remove moisture therefrom.

14. The container refilling system of claim 13, further comprising a controller configured to control the extent that the compressor compresses the cryogenic fluid based at least partially on the container temperature of the fluid container.

15. The container refilling system of claim 14, wherein the cooler is a fin pack cooler.

16. The container refilling system of claim 14, further comprising a vacuum source configured to evacuate moisture from the filter.

17. A method for filling a fluid container of a cryoablation system with a cryogenic fluid, the method comprising the steps of:
compressing and cooling the cryogenic fluid, wherein an extent of compression of the cryogenic fluid is based at least partially on a sensed container temperature of the fluid container;
delivering the cooled and compressed cryogenic fluid to a filter;
delivering the filtered, cooled and compressed cryogenic fluid to the fluid container; and
after delivering the filtered, cooled and compressed cryogenic fluid to the fluid container, selectively heating the filter to remove moisture from therefrom.

18. The method of claim 17, further comprising removing collected moisture from the filter under a vacuum.

19. The method of claim 17, further comprising analyzing, with a fluid analyzer, the compressed and cooled cryogenic fluid.

20. The method of claim 17, further comprising monitoring a water content of the compressed and cooled cryogenic fluid.
